(19) United States
(12) Patent Application Publication
(10) Pub. No.: US 2024/0355460 A1
(43) Pub. Date: Oct. 24, 2024

(54) SYSTEMS AND METHODS FOR IMPROVED PROVIDER PROCESSES USING CLAIM LIKELIHOOD RANKING

(71) Applicant: Optum Services (Ireland) Limited, Dublin (IE)

(72) Inventors: Rory SOBOLEWSKI, Dunkineely (IE); Dara FARRELLY, Milford (IE); Michael J. McCARTHY, Dublin (IE); Gavin ECCLES, Dublin (IE)

(73) Assignee: Optum Services (Ireland) Limited, Dublin (IE)

(21) Appl. No.: 18/304,788

(22) Filed: Apr. 21, 2023

(51) Int. Cl. G16H 40/20; G06N 5/022

(52) U.S. Cl. CPC G16H 40/20; G06N 5/022

(57) ABSTRACT

Systems and methods are disclosed for prioritizing one or more provider for data maintenance. The method includes receiving historical claim information from each of one or more providers. The method includes applying a respective model to the historical claim information received from each of the one or more providers. The method includes determining a respective expected number of claims for each of the one or more providers. The method includes normalizing the respective expected number of claims for each of the one or more providers. The method includes determining a respective claim likelihood score for each of the one or more providers. The method includes ranking one or more providers based on each provider's respective expected number of claims.

### TECHNICAL FIELD

Various embodiments of this disclosure relate generally to techniques for claim likelihood prediction, and, more particularly, to systems and methods for predicting a claim likelihood score based on historical provider data.

### BACKGROUND

In the healthcare industry, provider groups such as hospitals, medical practices, and other organizations typically comprise multiple individual healthcare service providers. These providers are maintained in a provider claims system, which is an essential component of the healthcare billing and reimbursement process. The provider claims system serves as the central repository of information for all providers within a provider group, facilitating the submission and processing of claims for healthcare services rendered to patients.

Managing the provider claims system incurs both financial and time costs, as each provider must be loaded into the system and their information regularly updated and maintained. This includes various outreach campaigns, data clean-up efforts, and system maintenance tasks that ensure the provider records are accurate and up-to-date.

However, recent analysis of provider claims systems has revealed that a significant proportion of providers, often exceeding 50%, have never submitted a claim. This phenomenon has several potential implications on provider operations, including, but not limited to:
Wasted resources: Maintaining providers who do not submit claims in the system represents an inefficient allocation of resources, as the associated costs may not yield any tangible benefit to the provider group;
Unaddressed systemic issues: Providers may not be submitting claims due to systemic or other issues that remain unidentified and unaddressed, ultimately impacting the financial health of the provider group; and
Inaccurate contract negotiations: Provider groups may negotiate contracts based on the number of providers within their organization, even though many of these providers may not submit claims. This can lead to inaccurate or unfavorable contract terms, which can have long-term negative consequences for the provider group.

In view of the aforementioned issues, there is a need for an improved system and method for managing healthcare provider claims, which can more effectively identify and address non-claiming providers, optimize resource allocation, and ensure more accurate contract negotiations.

This disclosure is directed to addressing above-referenced challenges. The background description provided herein is for the purpose of generally presenting the context of the disclosure. Unless otherwise indicated herein, the materials described in this section are not prior art to the claims in this application and are not admitted to be prior art, or suggestions of the prior art, by inclusion in this section.

### SUMMARY

The present disclosure solves this problem and/or other problems described above or elsewhere in the present disclosure and improves the state of conventional healthcare applications. The present disclosure teaches systems and methods for claim likelihood prediction using machine-learning.

In some aspects, the techniques described herein relate to a computer-implemented method for provider prioritization, including: receiving, by one or more processors, historical claim information from each of one or more providers; applying, by the one or more processors, a respective model to the historical claim information received from each of the one or more providers; determining, by the one or more processors, a respective expected number of claims for each of the one or more providers; normalizing, by the one or more processors, the respective expected number of claims for each of the one or more providers; determining, by the one or more processors, a respective claim likelihood score for each of the one or more providers; and ranking, by the one or more processors, one or more providers based on each provider's respective expected number of claims.

In some aspects, the techniques described herein relate to a system for provider prioritization, including: a memory storing instructions; and a processor executing the instructions to perform a process including: receiving historical claim information from each of one or more providers; applying a respective model to the historical claim information received from each of the one or more providers; determining a respective expected number of claims for each of the one or more providers; normalizing the respective expected number of claims for each of the one or more providers; determining a respective claim likelihood score for each of the one or more providers; and ranking one or more providers based on each provider's respective expected number of claims.

In some aspects, the techniques described herein relate to a computer implemented method for provider prioritization, including: receiving, by one or more processors, historical claim information from a plurality of providers, each provider of the plurality of providers belonging to a grouping of providers; applying, by the one or more processors, an Autoregressive Integrated Moving Average (ARIMA) model to the historical claim information from each of the plurality of providers, wherein each grouping of providers is associated with a respective model; determining, by the one or more processors, a respective expected number of claims for each of the plurality of providers; normalizing, by the one or more processors, the respective expected number of claims for each of the plurality of providers; determining, by the one or more processors, a respective claim likelihood score for each of the plurality of providers; categorizing, by the one or more processors, each of the plurality of providers within a claim likelihood score category; and prioritizing, by the one or more processors, one or more of the plurality of providers based on each provider's respective expected number of claims, wherein one or more bounds of each category adjust dynamically based at least in part on a population of providers.

It is to be understood that both the foregoing general description and the following detailed description are example and explanatory only and are not restrictive of the detailed embodiments, as claimed.

### BRIEF DESCRIPTION OF THE DRAWINGS

The accompanying drawings, which are incorporated in and constitute a part of this specification, illustrate various example embodiments and together with the description, serve to explain the principles of the disclosed embodiments.

FIG. 1 is a diagram showing an example of a system that is capable of claim prediction, according to some embodiments of the disclosure.

FIG. 2 is a diagram of example components of a claim likelihood platform, according to some embodiments of the disclosure.

FIG. 3 is a diagram of example components of a time series analysis module, according to some embodiments of the disclosure.

FIG. 4 is a flowchart for generating a claim likelihood score, according to some embodiments of the disclosure.

FIG. 5 is an illustrative example of the application of the claim likelihood platform to monthly provider data, according to some embodiments of the disclosure.

FIG. 6 depicts two graphs are presented that illustrate the transformation of raw predictions into normalized or transformed prediction, according to some embodiments of the disclosure.

FIG. 7 illustrates a claim likelihood score array, according to some embodiments of the disclosure.

FIG. 8 is an illustrative example of claim likelihood score stratification, according to some embodiments of the disclosure.

FIG. 9 is an illustrative example of a flowchart demonstrating a maintenance workflow, according to some embodiments of the disclosure.

FIG. 10 illustrates an implementation of a computer system that executes techniques presented herein, according to some embodiments of the disclosure.

### DETAILED DESCRIPTION

Various embodiments of this disclosure relate generally to techniques for claim likelihood prediction, and, more particularly, to systems and methods for predicting a claim likelihood score based on historical provider data.

As discussed above, the healthcare industry relies on provider claims systems to manage billing and reimbursement processes. However, a significant proportion of providers in these systems never submit claims, leading to wasted resources, unaddressed systemic issues, and inaccurate contract negotiations. Therefore, there is a need for an improved system and method for managing healthcare provider claims, which can effectively address non-claiming providers, optimize resource allocation, and ensure more accurate contract negotiations.

Motivated from the limitations of the conventional methodology, techniques disclosed herein provide a computer-implemented method for provider prioritization that addresses the problems and limitations by streamlining and optimizing the management of healthcare provider claims.

These techniques involve receiving historical claim information from each provider and applying a respective model to this data. By determining an expected number of claims for each provider and normalizing these numbers, the system can generate a claim likelihood score for each provider. Finally, the providers are ranked based on their respective expected number of claims.

These improvements offer several technical advantages, such as: enhanced resource allocation, whereby prioritizing and ranking providers based on their claim likelihood scores, the system helps to allocate resources more effectively, focusing on providers with higher claim activities and reducing wastage of resources on non-claiming providers; identification and resolution of systemic issues, whereby the disclosed techniques facilitate the identification of providers with low or no claim submissions, enabling the investigation of potential systemic issues that may be hindering the claim submission process, allowing for timely intervention and resolution of such issues, ultimately improving the financial health of the provider group; and more accurate contract negotiations, whereby providing a data-driven and comprehensive overview of provider claim activities, the system ensures more accurate contract negotiations, preventing unfavorable contract terms based on inflated provider numbers, leading to better long-term outcomes for the provider group.

Overall, the techniques disclosed herein enable a more efficient and effective management of healthcare provider claims by optimizing resource allocation, addressing systemic issues, and ensuring more accurate contract negotiations.

While principles of the present disclosure are described herein with reference to illustrative embodiments for particular applications, it should be understood that the disclosure is not limited thereto. Those having ordinary skill in the art and access to the teachings provided herein will recognize additional modifications, applications, embodiments, and substitution of equivalents all fall within the scope of the embodiments described herein. Accordingly, the invention is not to be considered as limited by the foregoing description.

Various non-limiting embodiments of the present disclosure will now be described to provide an overall understanding of the principles of the structure, function, and use of systems and methods disclosed herein for predicting a next text.

Reference to any particular activity is provided in this disclosure only for convenience and not intended to limit the disclosure. A person of ordinary skill in the art would recognize that the concepts underlying the disclosed devices and methods may be utilized in any suitable activity. The disclosure may be understood with reference to the following description and the appended drawings, wherein like elements are referred to with the same reference numerals.

The terminology used below may be interpreted in its broadest reasonable manner, even though it is being used in conjunction with a detailed description of certain specific examples of the present disclosure. Indeed, certain terms may even be emphasized below; however, any terminology intended to be interpreted in any restricted manner will be overtly and specifically defined as such in this Detailed Description section. Both the foregoing general description and the following detailed description are exemplary and explanatory only and are not restrictive of the features, as claimed.

In this disclosure, the term “based on” means “based at least in part on.” The singular forms “a,” “an,” and “the” include plural referents unless the context dictates otherwise. The term “exemplary” is used in the sense of “example” rather than “ideal.” The terms “comprises,” “comprising,” “includes,” “including,” or other variations thereof, are intended to cover a non-exclusive inclusion such that a process, method, or product that comprises a list of elements does not necessarily include only those elements, but may include other elements not expressly listed or inherent to such a process, method, article, or apparatus. The term “or” is used disjunctively, such that “at least one of A or B” includes, (A), (B), (A and A), (A and B), etc. Relative terms, such as, “substantially” and “generally,” are used to indicate a possible variation of +10% of a stated or understood value.

It will also be understood that, although the terms first, second, third, etc. are, in some instances, used herein to describe various elements, these elements should not be limited by these terms. These terms are only used to distinguish one element from another. For example, a first contact could be termed a second contact, and, similarly, a second contact could be termed a first contact, without departing from the scope of the various described embodiments. The first contact and the second contact are both contacts, but they are not the same contact.

As used herein, the term “if” is, optionally, construed to mean “when” or “upon” or “in response to determining” or “in response to detecting,” depending on the context. Similarly, the phrase “if it is determined” or “if [a stated condition or event] is detected” is, optionally, construed to mean “upon determining” or “in response to determining” or “upon detecting [the stated condition or event]” or “in response to detecting [the stated condition or event],” depending on the context.

Terms like “provider,” “merchant,” “vendor,” or the like generally encompass an entity or person involved in providing, selling, and/or renting items to persons such as a seller, dealer, renter, merchant, vendor, or the like, as well as an agent or intermediary of such an entity or person. An “item” generally encompasses a good, service, or the like having ownership or other rights that may be transferred. As used herein, terms like “user” or “customer” generally encompasses any person or entity that may desire information, resolution of an issue, purchase of a product, or engage in any other type of interaction with a provider. The term “browser extension” may be used interchangeably with other terms like “program,” “electronic application,” or the like, and generally encompasses software that is configured to interact with, modify, override, supplement, or operate in conjunction with other software.

As used herein, a “machine-learning model” generally encompasses instructions, data, and/or a model configured to receive input, and apply one or more of a weight, bias, classification, or analysis on the input to generate an output. The output may include, for example, a classification of the input, an analysis based on the input, a design, process, prediction, or recommendation associated with the input, or any other suitable type of output. A machine-learning model is generally trained using training data, e.g., experiential data and/or samples of input data, which are fed into the model in order to establish, tune, or modify one or more aspects of the model, e.g., the weights, biases, criteria for forming classifications or clusters, or the like. Aspects of a machine-learning model may operate on an input linearly, in parallel, via a network (e.g., a neural network), or via any suitable configuration.

The execution of the machine-learning model may include deployment of one or more machine-learning techniques, such as linear regression, logistical regression, random forest, gradient boosted machine (GBM), deep learning, and/or a deep neural network. Supervised and/or unsupervised training may be employed. For example, supervised learning may include providing training data and labels corresponding to the training data, e.g., as ground truth. Unsupervised approaches may include clustering, classification or the like. K-means clustering or K-Nearest Neighbors may also be used, which may be supervised or unsupervised. Combinations of K-Nearest Neighbors and an unsupervised cluster technique may also be used. Any suitable type of training may be used, e.g., stochastic, gradient boosted, random seeded, recursive, epoch or batch-based, etc.

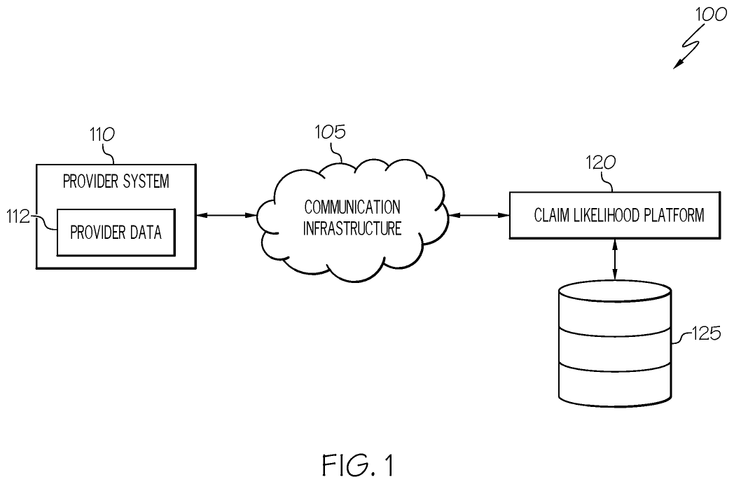

FIG. 1 is a diagram showing an example of a system that is capable of claim prediction, according to some embodiments of the disclosure. Referring to FIG. 1, a network environment 100 is depicted for claim likelihood assessment, in accordance with an embodiment of the present invention. The network environment 100 includes a communication infrastructure 105, a provider system 110, a claim likelihood platform 120, and a database 125.

In one embodiment, various elements of the network environment 100 communicate with each other through the communication infrastructure 105. The communication infrastructure 105 supports a variety of different communication protocols and communication techniques. In one embodiment, the communication infrastructure 105 allows the claim likelihood platform 120 to communicate with one or more other systems, including provider system 110, which is stored on a separate platform and/or system. The communication infrastructure 105 of the network environment 100 includes one or more networks such as a data network, a wireless network, a telephony network, or any combination thereof. It is contemplated that the data network is any local area network (LAN), metropolitan area network (MAN), wide area network (WAN), a public data network (e.g., the Internet), short range wireless network, or any other suitable packet-switched network, such as a commercially owned, proprietary packet-switched network, e.g., a proprietary cable or fiber-optic network, and the like, or any combination thereof. In addition, the wireless network is, for example, a cellular communication network and employs various technologies including 5G (5th Generation), 4G, 3G, 2G, Long Term Evolution (LTE), wireless fidelity (Wi-Fi), Bluetooth®, Internet Protocol (IP) data casting, satellite, mobile ad-hoc network (MANET), vehicle controller area network (CAN bus), and the like, or any combination thereof.

The provider system 110 includes one or more provider systems capable of sending provider data 115, such as data regarding claims and/or claim history. Claims can be insurance claims. The provider data 115 is managed and stored on one or more devices within the network environment 100, such as local or remote file servers, cloud-based storage services, or other forms of data repositories.

The claim likelihood platform 120 serves to intake and assess the provider data 115 and generate a claim likelihood score. The claim likelihood platform 120 can include various software applications, frameworks, or libraries that enable the processing and assessment of the provider data 115.

In one embodiment, the claim likelihood platform 120 is a platform with multiple interconnected components. The claim likelihood platform 120 includes one or more servers, intelligent networking devices, computing devices, components, and corresponding software for predicting and generating claim likelihood scores. In addition, it is noted that the claim likelihood platform 120 can be a separate entity of the system.

The database 125 is used to support the storage and retrieval of data related to the provider system 110, storing metadata about the provider data 115, such as claim type, date, and claim history, as well as any generated claim likelihood scores from the claim likelihood platform 120. The database 125 consists of one or more systems, such as a relational database management system (RDBMS), a NoSQL database, or a graph database, depending on the requirements and use cases of the network environment 100.

In one embodiment, the database 125 is any type of database, such as relational, hierarchical, object-oriented, and/or the like, wherein data are organized in any suitable manner, including data tables or lookup tables. In one embodiment, the database 125 accesses or includes any suitable data that is utilized to predict text. In one embodiment, the database 125 stores content associated with one or more system and/or platform, such as the claim likelihood platform 120 and manages multiple types of information that provide means for aiding in the content provisioning and sharing process. The database 125 includes various information related to documents, topics, and the like. It is understood that any other suitable data can be included in the database 125.

In one embodiment, the database 125 includes a machine-learning based training database with a pre-defined mapping defining a relationship between various input parameters and output parameters based on various statistical methods. For example, the training database includes machine-learning algorithms to learn mappings between input parameters related to provider data 115. In an embodiment, the training database is routinely updated and/or supplemented based on machine-learning methods.

The claim likelihood platform 120 communicates with other components of the communication infrastructure 105 using well known, new or still developing protocols. In this context, a protocol includes a set of rules defining how the network nodes within the communication infrastructure 105 interact with each other based on information sent over the communication links. The protocols are effective at different layers of operation within each node, from generating and receiving physical signals of various types, to selecting a link for transferring those signals, to the format of information indicated by those signals, to identifying which software application executing on a computer system sends or receives the information. The conceptually different layers of protocols for exchanging information over a network are described in the Open Systems Interconnection (OSI) Reference Model.

Communications between the network nodes are typically effected by exchanging discrete packets of data. Each packet typically comprises (1) header information associated with a particular protocol, and (2) payload information that follows the header information and contains information that is processed independently of that particular protocol. In some protocols, the packet includes (3) trailer information following the payload and indicating the end of the payload information. The header includes information such as the source of the packet, its destination, the length of the payload, and other properties used by the protocol. Often, the data in the payload for the particular protocol includes a header and payload for a different protocol associated with a different, higher layer of the OSI Reference Model. The header for a particular protocol typically indicates a type for the next protocol contained in its payload. The higher layer protocol is said to be encapsulated in the lower layer protocol. The headers included in a packet traversing multiple heterogeneous networks, such as the Internet, typically include a physical (layer 1) header, a data-link (layer 2) header, an internetwork (layer 3) header and a transport (layer 4) header, and various application (layer 5, layer 6 and layer 7) headers as defined by the OSI Reference Model.

In operation, the network environment 100 provides a framework for processing and analyzing large amounts of claim-related content, leveraging the capabilities of natural language processing and database technologies to support a wide range of use cases and applications. For example, the network environment 100 can be used to perform claim classification, such as identifying whether a given claim is related to property, health, or automotive insurance. The network environment 100 can also be used to extract key information from the provider data 115, or to predict the likelihood of future claims within one or more claims data sets.

To perform these tasks, the claim likelihood platform 120 utilizes various techniques, such as named entity recognition, which identifies and categorizes named entities in the provider data 115, such as people, organizations, or locations. The claim likelihood platform 120 can also utilize data modeling techniques, which identify and extract the main themes or topics discussed in the data related to claims or claim history.

To support the storage and retrieval of data related to the provider data 115, the database 125 is used to store metadata about the claims, such as claimant, date, and claim type. The database 125 can also be used to store any extracted information from the claim likelihood platform 120, such as named entities or patterns identified in the provider data 115.

In addition to these use cases, the network environment 100 can be used to support a wide range of other applications and tasks, such as search and recommendation systems, data summarization, and data visualization. For example, the network environment 100 is used to build a search engine that enables users to search for specific keywords or phrases within the provider data 115, returning a list of relevant claims and information about the contexts in which the keywords or phrases appear.

In the context of the present invention, provider data 115 refers to a collection of data related to claims and claim history that are associated with one or more providers. For example, the provider data 115 can be related to a specific type of insurance, industry, or domain. Each data entry within the provider data 115 can be associated with one or more providers, which includes insurance companies, organizations, or entities that created or contributed to the claim.

To facilitate the association of the provider data 115 with one or more providers, the network environment 100 utilizes various techniques, such as metadata extraction, data processing, or user input. For example, metadata extraction involves extracting information about the claimant, date of the claim, and the source of the claim, which is then be used to associate the claim with one or more providers. Data processing involves analyzing the content of the claim to identify entities or themes that are associated with particular providers. User input involves allowing users to manually specify the provider of a claim or data entry, either during the initial ingestion process or at a later time.

Once the association between the provider data 115 and one or more providers has been established, this information can be used in various ways within the network environment 100. For example, users are able to search for claims based on the provider, allowing them to quickly find relevant information related to a particular insurance company or organization. Additionally, the provider information is used to help users identify the credibility or reliability of the information contained within the claims.

In addition to being associated with one or more providers, each entry in the provider data 115 can also be associated with one or more topics. The topic of a claim or data entry can be defined in various ways, such as based on the content of the claim, the domain or industry it relates to, or user-defined tags or categories.

To facilitate the association of provider data 115 with one or more claim-related categories, the network environment 100 utilizes various techniques, such as claim type classification, keyword extraction, or user input. For example, claim type classification involves identifying the main themes or types of claims within the provider data 115, and using these categories to associate the data with one or more claim-related categories. Keyword extraction involves identifying important keywords or phrases within the claim data, and using these keywords to associate the data with one or more categories. User input involves allowing users to manually specify the category of a claim or data entry, either during the initial intake process or at a later time.

Once the association between the provider data 115 and one or more claim-related categories has been established, this information is used in various ways within the network environment 100. For example, users are able to search for claims or claim data based on category, allowing them to quickly find relevant information related to a particular claim type or theme. Additionally, the category information is used to help users analyze and understand the content of the claims, by identifying the main themes or types of claims present within the provider data 115.

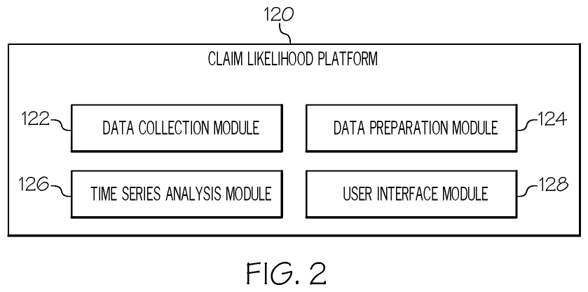

FIG. 2 is a diagram of example components of a claim likelihood platform, according to some embodiments of the disclosure. Referring to FIG. 2, the claim likelihood platform 120 is a component of the network environment 100. The claim likelihood platform 120 provides the processing capabilities necessary to analyze and extract information from the provider data 115 and/or one or more claim therein. As used herein, terms such as “component” or “module” generally encompass hardware and/or software, e.g., that a processor or the like is used to implement associated functionality. By way of example, the claim likelihood platform 120 includes one or more components for predicting and/or detecting the likelihood of a claim and, in some embodiments, thereby generating a claim likelihood score. It is contemplated that the functions of these components are combined in one or more components or performed by other components of equivalent functionality. The claim likelihood platform 120 includes one or more modules, such as a data collection module 122, a data preparation module 124, a time-series analysis module 126, and a user interface module 128, or any combination thereof.

In one embodiment, the data collection module 122 collects relevant data, e.g., claim data, and the like, through various data collection techniques. In one embodiment, the data collection module 122 uses a web-crawling component to access various databases, e.g., the database 125, or other information sources, e.g., any third-party databases, to collect relevant data associated with one or more claim, such as provider data 115. In one embodiment, the data collection module 122 includes various software applications, e.g., data mining applications in Extended Meta Language (XML), which automatically search for and return relevant data. The data collection module 122, in some embodiments, is responsible for in-taking one or more documents, claims, or data associated with a claim, into the claim likelihood platform 120. The data collection module 122 is designed to work with various types of claim data, such as text-based documents, images, audio, or video. The module is designed to accept documents in various formats, such as plain text, PDF, HTML, XML, or other structured or unstructured data formats.

Once the claim data has been collected, the data preparation module 124 processes the claim data into a format which may be used as an input to one or more module, such as tokens that can be used as input to one or more time-series analysis processing algorithms. The data preparation module 124 uses various techniques to tokenize the claim data, such as breaking the text strings into individual words, removing stop words, converting the words to their base form (e.g., stemming), or otherwise transforming the claim data into a standardized encoding format which is suitable for processing by one or more model, such as a time-series analysis model. The data preparation module 124 is responsible for identifying important entities within the documents, such as people, places, or organizations. In one example embodiment, the data preparation module 124 examines the collected data for any errors to eliminate bad data, e.g., redundant, incomplete, or incorrect data, to create high-quality data. In one example embodiment, collected data, e.g., raw data, is converted into a common format, e.g., machine readable form, that is easily processed by other modules and platforms.

The time-series analysis module 126 is responsible for applying time-series analysis techniques to the provider data 115 and generating insights and information from the data. The time-series analysis module 126 includes various algorithms and techniques, such as autoregressive integrated moving average (ARIMA), exponential smoothing state space model (ETS), and seasonal decomposition of time-series (STL). The time-series analysis module 126 also includes advanced techniques, such as Bayesian structural time-series models, to improve the accuracy and performance of the time-series analysis algorithms. Moreover, the time-series analysis module 126 includes one or more machine-learning models and/or techniques, including natural language processing, to generate insights and information from the provider data 115.

The time-series analysis module 126 is configured to employ various types of time-series analysis techniques for processing and analyzing provider data 115 to generate insights and information. The techniques include decomposition methods, such as Seasonal Decomposition of Time-series (STL) and Classical Decomposition, which focus on breaking down a time-series into constituent components like trend, seasonality, and irregular fluctuations. Smoothing methods, such as Simple Moving Average (SMA), Exponential Moving Average (EMA), Exponential Smoothing State Space Model (ETS), and Holt-Winters Exponential Smoothing, are utilized to smooth out noise and irregular fluctuations in the time-series to reveal underlying patterns and trends. Autoregressive methods, including Autoregressive Model (AR), Moving Average Model (MA), Autoregressive Integrated Moving Average (ARIMA), Seasonal ARIMA (SARIMA), and Autoregressive Fractionally Integrated Moving Average (ARFIMA), model the time-series based on its own past values. State space methods, like Kalman Filter and Bayesian Structural Time-series (BSTS), use a state space representation of the time-series data, incorporating both the observed data and hidden states to model the underlying dynamic process. Machine learning and deep learning methods, such as Long Short-Term Memory (LSTM) Neural Networks, Gated Recurrent Unit (GRU) Neural Networks, Convolutional Neural Networks (CNN) for Time-series, Deep Belief Networks (DBN), Echo State Networks (ESN), and Prophet, are used to leverage algorithms to model complex patterns in time-series data. Ensemble methods, including model averaging, stacking, bagging, and boosting, combine multiple time-series models to achieve better forecasting performance. Other specialized techniques, like Dynamic Time Warping (DTW), Wavelet Analysis, and Fourier Analysis, are also employed to address specific time-series challenges. The time-series analysis module 126 utilizes one or a combination of these techniques, depending on the characteristics of the provider data 115 and the particular analysis objectives.

In one embodiment, the claim likelihood platform 120 is configured for unsupervised time-series analysis that does not require training using known outcomes. The unsupervised time-series analysis utilizes algorithms to analyze and cluster unlabeled datasets and discover hidden patterns or data groupings, e.g., similarities and differences within provider data 115, without supervision. In one example embodiment, the unsupervised time-series analysis implements approaches that include clustering (e.g., deep embedded clustering, K-means clustering, hierarchical clustering, and probabilistic clustering), association rules, classification, principal component analysis (PCA), or the like.

In one embodiment, the claim likelihood platform 120 is also configured for supervised time-series analysis that utilizes training data for training a time-series analysis model configured to predict and/or detect next data points based on the relevant provider data 115. In one example embodiment, the claim likelihood platform 120 performs model training using training data, e.g., data from other modules that contains input and correct output, to allow the model to learn over time. The training is performed based on the deviation of a processed result from a documented result when the inputs are fed into the time-series analysis model, e.g., an algorithm measures its accuracy through the loss function, adjusting until the error has been sufficiently minimized. In one embodiment, the claim likelihood platform 120 randomizes the ordering of the training data, visualizes the training data to identify relevant relationships between different variables, identifies any data imbalances, and splits the training data into two parts where one part is for training a model and the other part is for validating the trained model, de-duplicating, normalizing, correcting errors in the training data, and so on. The claim likelihood platform 120 implements various time-series analysis techniques, e.g., ARIMA, state space models, Fourier analysis, wavelet analysis, etc.

In one embodiment, the provider system 110 implements time-series analysis techniques to analyze, understand, and derive insights from the provider data 115. Time-series analysis is applied to analyze data points collected over time, allowing for the identification of patterns, trends, and seasonality, enabling real-world applications such as forecasting, anomaly detection, and trend analysis. In one embodiment, time-series analysis generally encompasses techniques including, but not limited to, decomposition, smoothing, differencing, and modeling. The user interface module 128 provides a way for users to interact with the claim likelihood platform 120, allowing them to configure and customize the natural language processing algorithms, and to view the results of the analysis. The user interface module 128 includes various features, such as search capabilities, data visualization tools, and customization options.

In one embodiment, the user interface module 128 enables a presentation of a graphical user interface (GUI) that facilitates claim visualizations, such as claim prediction visualization. The user interface module 128 employs various application programming interfaces (APIs) or other function calls corresponding to one or more application, thus enabling the display of graphics primitives such as icons, bar graphs, menus, buttons, data entry fields, etc. In another embodiment, the user interface module 128 causes interfacing of guidance information to include, at least in part, one or more annotations, audio messages, video messages, or a combination thereof pertaining to one or more notification. In another example embodiment, the user interface module 128 operates in connection with augmented reality (AR) processing techniques, wherein various applications, graphic elements, and features interact to present one or more notifications in a format that is understandable by the recipients, e.g., service providers.

In addition to the modules described above, the claim likelihood platform 120 also includes various sub-modules, such as data preprocessing modules, feature extraction modules, and model selection modules. These sub-modules are used to preprocess the data before it is passed to the time-series analysis module 126, extract important features from the data, and select the most appropriate machine-learning model for the task at hand.

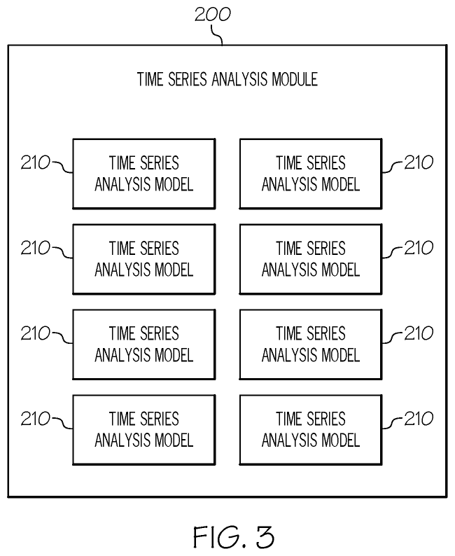

FIG. 3 is a diagram of example components of a time series analysis module, according to some embodiments of the disclosure. Referring to FIG. 3, a time-series analysis module 200 is shown. The time-series analysis module 200 can be the same as the time-series analysis module 126 shown in FIG. 2, or it can be a separate model. The time-series analysis module 200 is a sophisticated system designed for processing and analyzing provider data 115 to generate insights and predictions related to claims, such as the number of predicted claims over a set period of time. The module 200 comprises one or more time-series analysis models 210, each of which are associated with a single claim provider, a grouping of claim providers, or other relevant categorizations.

Each time-series analysis model 210 is tailored to intake a portion of provider data 115 and process it using various time-series analysis techniques, such as decomposition, smoothing, autoregressive methods, state space methods, machine learning and deep learning methods, ensemble methods, and other specialized techniques. The choice of techniques employed by a specific model 210 are determined by the characteristics of the provider data 115, the nature of the associated claim providers, and the objectives of the analysis.

To ensure accurate and robust predictions, each time-series analysis model 210 undergoes a training phase using historical data, which includes known outcomes. This training phase enables the model to learn patterns, trends, and seasonality in the provider data 115, as well as any underlying relationships between variables. The model 210 can be validated against a separate dataset to evaluate its performance and make necessary adjustments before deployment.

Once the time-series analysis model 210 is trained and validated, it can intake new provider data 115 and generate outputs indicative of predicted claims. These outputs can include the number of predicted claims over a set period of time or other relevant metrics, such as the likelihood of a claim being filed or the potential monetary value of a claim. The model 210 also provides confidence intervals or uncertainty estimates for its predictions, which can be useful for risk assessment and decision-making purposes.

To maintain optimal performance, the time-series analysis model 210 is periodically updated or retrained with new data, allowing it to adapt to changing trends and patterns in the provider data 115. Additionally, the module 200, in some embodiments, employs multiple time-series analysis models 210 in parallel or as an ensemble, combining their outputs to generate more accurate and robust predictions.

In summary, the time-series analysis module 200, comprising one or more time-series analysis models 210, plays an important role in processing provider data 115 to generate insights and predictions related to claims. By leveraging a diverse range of time-series analysis techniques and continuously updating the models with new data, the module 200 can provide valuable outputs, such as the number of predicted claims over a set period, which can inform decision-making and risk management strategies for claim providers and other stakeholders.

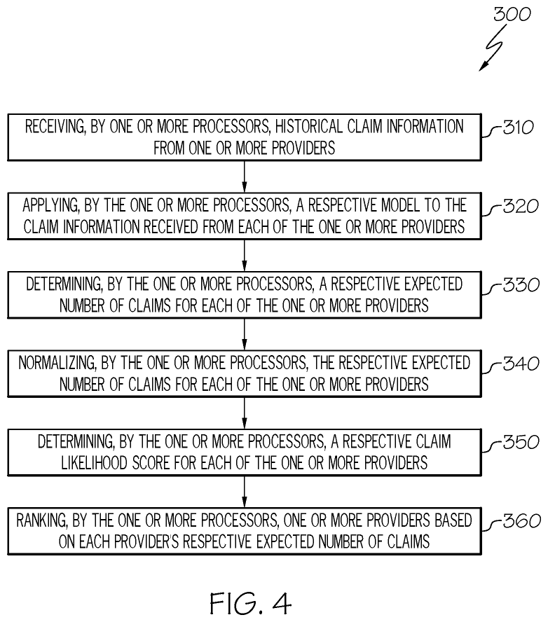

FIG. 4 is a flowchart for generating a claim likelihood score, according to some embodiments of the disclosure. Referring to FIG. 4, a method 300 for generating a claim likelihood score is provided. In one embodiment, the method 300 is performed by the claim likelihood platform 120, or one or more components therein such as, e.g., the data collection module 122, the data preparation module 124, the time series analysis module 126, and the user interface module 128. At step 310, the method includes receiving historical claim information from one or more provider. In an embodiment of the invention, the data collection module 122 is configured to receive historical claim information from one or more providers. The network environment 100 facilitates communication between the provider system 110 and the claim likelihood platform 120. The provider system 110 stores provider data 115, which can include historical claim information related to a plurality of claims.

The historical claim information comprises various types of data associated with the claims, such as the claim amount, claim date, claim type, policyholder information, or the like. The data collection module 122 can establish a secure connection with the provider system 110 through the communication infrastructure 105, thereby enabling the transfer of the historical claim information from the provider system 110 to the data collection module 122. The secure connection is implemented using various security protocols, such as Secure Socket Layer (SSL), Transport Layer Security (TLS), or the like, to ensure the confidentiality and integrity of the historical claim information during transmission.

In some embodiments, the data collection module 122 receives historical claim information from multiple providers. The multiple providers include different types of insurance providers, such as health insurance providers, automobile insurance providers, property insurance providers, or the like, or can include a plurality of similar types of insurance providers, such as receiving data from multiple health insurance providers. Moreover, the data received can be grouped, such that one or more health insurance providers are considered part of a first grouping. Receiving historical claim information from multiple providers facilitates a more comprehensive analysis of claim patterns and trends across different sectors of the insurance industry, thereby enhancing the accuracy of the claim likelihood predictions generated by the claim likelihood platform 120.

The data collection module 122 furthers include a data validation component that is configured to validate the received historical claim information. The data validation component performs various checks on the received data, such as checking for missing data, inconsistencies, duplicates, or the like. In instances where the data validation component identifies issues with the received historical claim information, the data collection module 122 requests additional or corrected data from the provider system 110 or can apply data imputation techniques to address the identified issues.

At step 320, the method includes applying a respective model to the claim information from each one or more providers. In an embodiment of the invention, the data preparation module 124 is configured to process and prepare the received historical claim information for subsequent analysis by the time-series analysis module 126. The data preparation module 124 applies various techniques to the historical claim information, such as data cleaning, normalization, transformation, or the like, to generate a processed claim dataset suitable for modeling purposes.

The time-series analysis module 126 is responsible for generating and applying respective models to the processed claim dataset associated with each provider. The models are tailored to address the specific characteristics and features of the historical claim information corresponding to each provider, such as claim frequency, claim severity, seasonal patterns, or the like. These models are based on various statistical, machine learning, or artificial intelligence techniques, such as autoregressive integrated moving average (ARIMA) models, exponential smoothing state space models, artificial neural networks, support vector machines, random forests, or the like.

In some embodiments, the time-series analysis module 126 employs a model selection process to determine the most suitable model for each provider's historical claim information. The model selection process involves evaluating the performance of multiple candidate models based on various performance metrics, such as mean absolute error (MAE), mean squared error (MSE), root mean squared error (RMSE), mean absolute percentage error (MAPE), or the like. The model with the best performance, as indicated by the chosen performance metrics, is selected as the respective model for the particular provider.

Once the respective model has been selected and trained on the processed claim dataset associated with a particular provider, the time-series analysis module 126 applies the respective model to the claim information from the corresponding provider. This application involves inputting the processed claim dataset into the selected model, which then generates claim likelihood predictions based on the underlying patterns and trends identified within the historical claim information. These claim likelihood predictions are utilized by the claim likelihood platform 120 to produce claim likelihood scores, as further described in subsequent steps.

In embodiments where the data collection module 122 receives historical claim information from multiple providers, the time-series analysis module 126 generates and applies respective models to the claim information from each provider separately. This approach ensures that the unique characteristics and trends associated with each provider's historical claim information are accounted for in the claim likelihood predictions, thereby improving the overall accuracy and relevance of the generated claim likelihood scores.

In some embodiments, each provider, or grouping of providers, has their own respective models tailored to address the specific characteristics and features of the historical claim information associated with the provider or provider group. This customization allows for more accurate and relevant claim likelihood predictions that consider the unique patterns, trends, and factors influencing claims within each provider or provider group.

When processing historical claim information from multiple providers or provider groups, the data preparation module 124 organizes the data into separate datasets corresponding to each provider or provider group. This organization enables the time-series analysis module 126 to independently analyze and model the historical claim information for each provider or provider group, thereby capturing their distinct claim patterns and trends.

For each provider or provider group, the time-series analysis module 126 generates and evaluates multiple candidate models based on various statistical, machine learning, or artificial intelligence techniques, as previously mentioned. The selection of the most suitable model for each provider or provider group is guided by the performance metrics mentioned earlier, such as MAE, MSE, RMSE, or MAPE.

Upon selecting the best-performing model for each provider or provider group, the time-series analysis module 126 trains the respective models on the processed claim datasets associated with the corresponding providers or provider groups. The trained models are then be applied to the claim information from their respective providers or provider groups to generate claim likelihood predictions that accurately reflect the unique claim characteristics and trends specific to each provider or provider group.

The utilization of separate models for individual providers or provider groups ensures a higher degree of accuracy and relevance in the generated claim likelihood predictions. This approach accounts for the potential differences in claim patterns, claim handling processes, policyholder demographics, or other factors that may influence claim likelihood across various providers or provider groups. Consequently, the claim likelihood platform 120 can produce more accurate and reliable claim likelihood scores, thereby enabling more informed decision-making for insurance providers and other stakeholders in the insurance industry.

At step 330, the method includes forecasting a respective expected number of claims for each one or more providers. In an embodiment of the invention, the time-series analysis module 126 is configured to perform the forecasting based on the claim likelihood predictions generated by the respective models 210 applied to the claim information from each provider or provider group. In some embodiments, the claim likelihood predictions form a portion of or all the forecasted expected number of claims.

The forecasting process involves aggregating the claim likelihood predictions over a specified time horizon, such as a month, quarter, or year, to generate the respective expected number of claims for each provider or provider group. This aggregation is performed using various techniques, such as summation, averaging, weighted averaging, or the like, depending on the specific requirements and characteristics of the historical claim information and the respective models 210.

In some embodiments, the time-series analysis module 126 generates confidence intervals or prediction intervals for the expected number of claims, which provide an indication of the range within which the actual number of claims is likely to fall. These intervals are derived using various statistical techniques, such as bootstrapping, Monte Carlo simulation, or the like, and are based on the underlying assumptions and uncertainties associated with the respective models 210 and the historical claim information.

The forecasted expected number of claims for each provider or provider group are stored in the database 125 for subsequent retrieval, analysis, and presentation by the claim likelihood platform 120. In particular, the user interface module 128 are configured to display the forecasted expected number of claims, along with any associated confidence or prediction intervals, to users of the claim likelihood platform 120.

In addition to displaying the forecasted expected number of claims, the user interface module 128 also provides various visualization tools and features to facilitate the comparison and analysis of the forecasted results across different providers or provider groups. For example, the user interface module 128 generates charts, graphs, tables, or the like, illustrating the forecasted expected number of claims over time, by claim type, by geographical region, or other relevant dimensions. These visualizations enable users to easily identify trends, patterns, and potential areas of concern or opportunity related to the expected number of claims.

Furthermore, the user interface module 128 provides interactive features that allow users to adjust the parameters, assumptions, or time horizons used in the forecasting process, thereby enabling users to explore various “what-if” scenarios and evaluate the potential impact of different factors on the expected number of claims. Such interactive features include sliders, drop-down menus, or other input controls that allow users to modify the underlying data or model parameters and instantly view the resulting changes in the forecasted expected number of claims.

In some embodiments, the claim likelihood platform 120 continuously updates the forecasted expected number of claims as new historical claim information becomes available from the provider system 110 or as the respective models 210 are refined and improved over time. In this manner, the claim likelihood platform 120 provides insurance providers and other stakeholders with timely, accurate, and actionable insights into the expected number of claims, thereby enabling more effective risk management, data maintenance, pricing, and claim handling strategies within the insurance industry.

At step 340, the method further includes normalizing each respective expected number of claims. In an embodiment of the invention, the data preparation module 124 is configured to perform normalization on the forecasted expected number of claims generated by the time-series analysis module 126 for each provider or provider group.

Normalization is a process that aims to standardize the forecasted expected number of claims across different providers or provider groups, facilitating comparisons and analyses between them. This process involves transforming the raw forecasted expected number of claims into a normalized metric or score that takes into account various factors, such as the size of the provider or provider group, the number of policyholders, the total amount of premiums, or the like.

There are several normalization techniques that can be employed, such as min-max scaling, z-score normalization, or percentile normalization, among others. The choice of normalization technique depends on the specific requirements, characteristics, and assumptions of the claim likelihood platform 120 and the underlying historical claim information.

For example, min-max scaling may involve linearly transforming the forecasted expected number of claims for each provider or provider group, such that the minimum value corresponds to a predetermined lower bound (e.g., 0) and the maximum value corresponds to a predetermined upper bound (e.g., 1). This transformation may be performed using the following formula:

Normalized_value=(Raw_value−Min_value)/(Max_value−Min_value)

where Normalized_value represents the normalized expected number of claims, Raw_value denotes the raw forecasted expected number of claims, and Min_value and Max_value represent the minimum and maximum values of the expected number of claims across all providers or provider groups, respectively.

Z-score normalization, on the other hand, may involve transforming the forecasted expected number of claims for each provider or provider group, such that the resulting values have a mean of 0 and a standard deviation of 1. This transformation may be performed using the following formula:

Normalized_value=(Raw_value−Mean)/Standard_deviation

where Normalized_value represents the normalized expected number of claims, Raw_value denotes the raw forecasted expected number of claims, Mean is the average of the expected number of claims across all providers or provider groups, and Standard_deviation represents the standard deviation of the expected number of claims across all providers or provider groups.

Once the normalization process is complete, the normalized expected number of claims is stored in the database 125 for subsequent retrieval, analysis, and presentation by the claim likelihood platform 120. In particular, the user interface module 128 is configured to display the normalized expected number of claims, along with any associated visualizations, comparisons, or other analytical tools, to users of the claim likelihood platform 120, such as insurance providers, regulators, or other stakeholders in the insurance industry.

The utilization of normalized expected number of claims enables more meaningful comparisons and insights across different providers or provider groups, accounting for variations in size, scope, or other factors that may influence claim likelihood. Consequently, the claim likelihood platform 120 can provide more accurate and actionable information for insurance providers and other stakeholders in the insurance industry, facilitating more informed decision-making in areas such as risk management, data maintenance, pricing, and claim handling strategies.

At step 350, the method includes determining a claim likelihood score for each one or more providers. The method includes a stratification of the normalized expected number of claims. The determination and/or stratification includes receiving a set of normalized number (expected number of claims), determining one or more score categories (such as a claim likelihood score category), and allocating one or more of the normalized numbers into the respective score category based on one or more predefined criteria.

In some embodiments, the results of the normalization are utilized as a claim likelihood score for each provider. For example, each provider has their forecasting claim predictions normalized by one or more methods and/or calculations as discussed herein, and the output of the normalization for a forecast period is set and/or utilized as the claim likelihood score. It will be appreciated the selection and/or determination of the claim likelihood scores can involve applying one or more models which are unique to each provider, or can involve applying a model to a portion of the providers while applying one or more models to one or more other providers.

At step 360, the method includes prioritizing one or more providers based on each provider's respective expected number of claims and/or on a claim likelihood score associated with each provider and/or a claim likelihood score category associated with each provider. In an embodiment of the invention, the user interface module 128 is configured to facilitate the prioritization process by presenting the relevant information to the users of the claim likelihood platform 120.

The prioritization process can involve ranking the providers or provider groups based on their respective normalized expected number of claims, claim likelihood scores, or claim likelihood score categories. The ranking is performed in ascending or descending order, depending on the specific requirements, goals, or preferences of the users or the claim likelihood platform 120.

In some embodiments, the claim likelihood scores are calculated based on the normalized expected number of claims, as well as additional factors or metrics that may be relevant to the insurance industry, such as claim severity, claim frequency, policyholder demographics, or the like. These additional factors or metrics are incorporated into the calculation of the claim likelihood scores through various weighting schemes or aggregation methods, depending on the specific assumptions, requirements, or characteristics of the claim likelihood platform 120 and the underlying historical claim information.

In other embodiments, the claim likelihood score categories are established by dividing the range of possible claim likelihood scores into discrete intervals or groups, each corresponding to a specific level of claim likelihood. These categories are defined using various criteria, such as percentiles, standard deviations, or other statistical measures, depending on the specific needs or goals of the users or the claim likelihood platform 120.

Once the providers or provider groups have been prioritized based on their respective expected number of claims, claim likelihood scores, or claim likelihood score categories, the resulting rankings are presented to the users of the claim likelihood platform 120 through various visualization techniques, such as tables, charts, graphs, or other graphical representations. These visualizations are designed to facilitate the interpretation, comparison, and analysis of the prioritized providers or provider groups, enabling users to make more informed decisions in areas such as risk management, pricing, data maintenance, claim handling strategies, or the like.

The user interface module 128 can also provide various interactive features or tools that allow users to customize the prioritization process, such as adjusting the weighting schemes, aggregation methods, or criteria used to calculate the claim likelihood scores or define the claim likelihood score categories. These customization options enable users to tailor the prioritization process to their specific needs, preferences, or assumptions, thereby increasing the accuracy, relevance, and utility of the resulting rankings and insights.

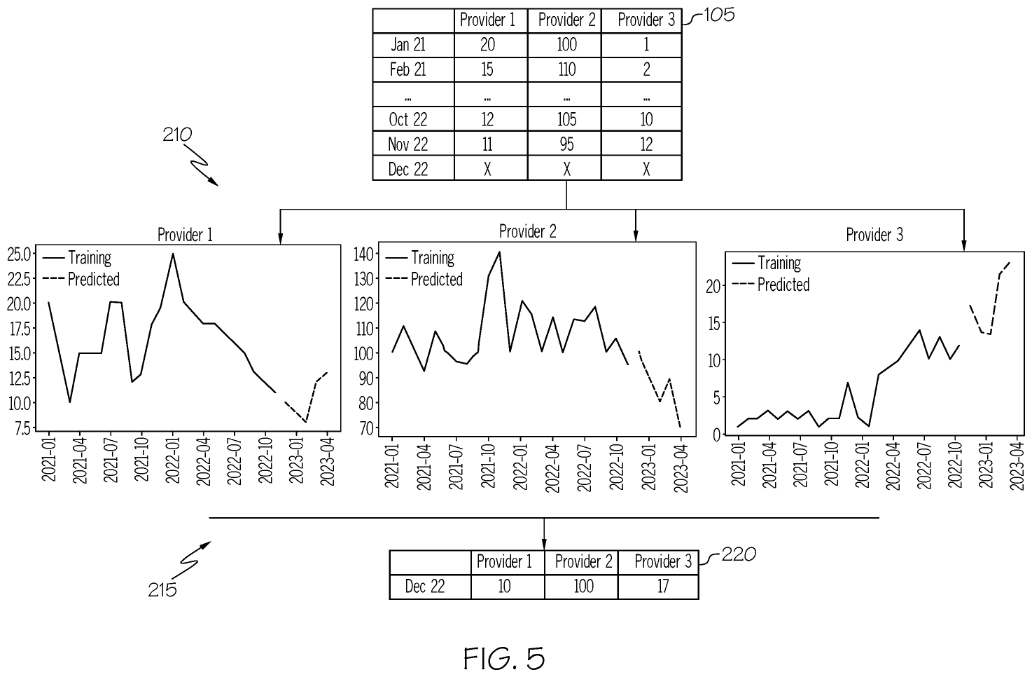

FIG. 5 is an illustrative example of the application of the claim likelihood platform to monthly provider data, according to some embodiments of the disclosure. The figure depicts an array containing monthly provider data 115 for three providers, the application of one or more time-series analysis models 210 to each provider, a time-series analysis model output 215 for each provider, and a prediction array 220, which contains predictions of the number of claims for each provider for a given time frame.

The monthly provider data 115 for three providers is represented in the form of an array, with each column corresponding to a specific provider and each row representing a particular month's claim data. This data includes information such as claim count, claim type, claim amount, and other relevant claim-related data. The data collection module 122 retrieves this monthly provider data 115 from the provider system 110, processes it, and prepares it for further analysis.

Next, one or more time-series analysis models are applied to the monthly provider data 115 for each provider. These models are based on various statistical, machine learning, or artificial intelligence techniques, as previously discussed, and tailored to address the specific characteristics and features of the historical claim information corresponding to each provider.

After applying the time-series analysis models to the monthly provider data 115, the time-series analysis module 126 generates a time-series analysis model output 215 for each provider. The model outputs 215 includes the results of the analysis, such as claim likelihood predictions, claim frequency patterns, seasonal trends, or other relevant information derived from the historical claim data.

The prediction array 220 contains the predictions of the number of claims for each provider for a given time frame, which are based on the time-series analysis model output 215. Each entry in the prediction array 220 corresponds to a specific provider and represents the expected number of claims for that provider within the specified time frame. These predictions can be used to inform various aspects of the insurance industry, such as risk management, data maintenance, pricing, claim handling strategies, or the like.

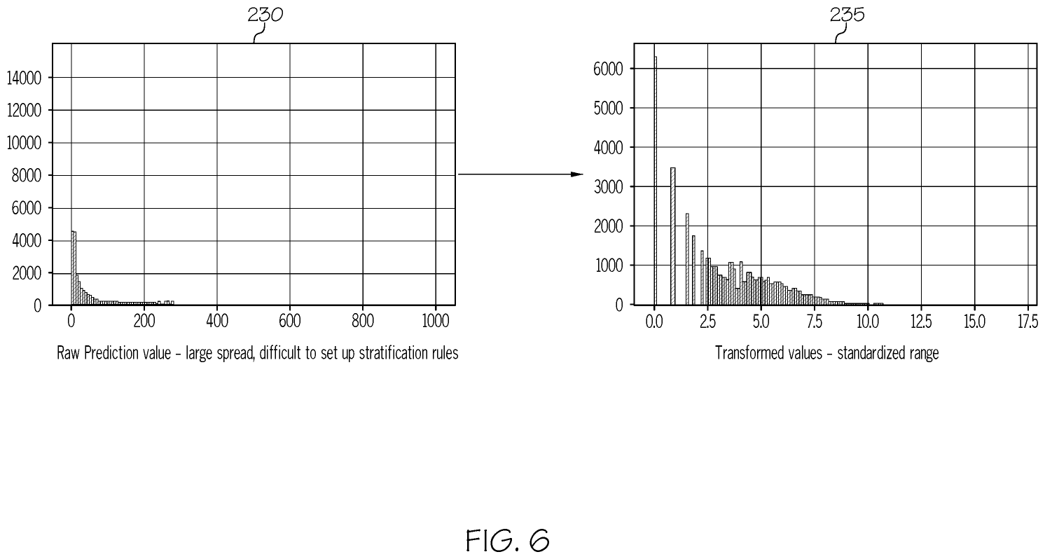

FIG. 6 depicts two graphs that illustrate the transformation of raw predictions into normalized or transformed prediction, according to some embodiments of the disclosure. Referring to FIG. 6, two graphs are presented that illustrate the transformation of raw predictions 230 into normalized or transformed predictions 235 for each provider. These graphs provide a visual representation of the claim likelihood scores before and after normalization, allowing for more effective and efficient stratification and prioritization of providers.

The first graph is a bar graph showing the raw prediction 230 for every provider. This graph represents the expected number of claims for each provider as determined by the claim likelihood platform 120, based on the application of respective time-series analysis models to each provider's historical claim data. As can be observed, the raw predictions 230 may exhibit a large spread of values, which may make it difficult or inefficient to apply stratification rules or to meaningfully compare the claim likelihood scores among providers.

The second graph depicts the transformed predictions 235, which show the normalized and/or transformed values for each provider over a standardized range. In this example, the standardized range is shown to be 0 to 17.5. The normalization process, performed by the normalization module 129, adjusts the raw predictions 230 so that they fall within the standardized range, allowing for easier comparison and prioritization of providers based on their respective claim likelihood scores.

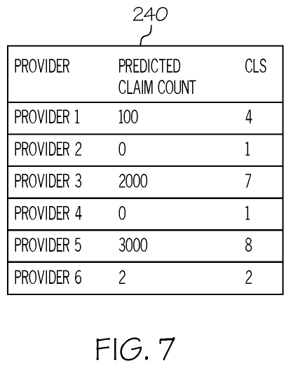

FIG. 7 illustrates a claim likelihood score array, according to some embodiments of the disclosure. Referring to FIG. 7, a claim likelihood score array 240 is presented, which provides a visual representation of the claim likelihood scores for each provider in a tabular format. Each row of the claim likelihood score array 240 represents a provider, and the columns display data for a raw predicted claim count and a claim likelihood score. The first column in the claim likelihood score array 240 corresponds to the raw predicted claim count for each provider. This value represents the expected number of claims for the respective provider as determined by the claim likelihood platform 120, based on the application of the time-series analysis models to the provider's historical claim data. The second column in the claim likelihood score array 240 presents the claim likelihood score for each provider. This score is derived from the normalized or transformed predictions, as discussed in relation to FIG. 6. The claim likelihood score is a standardized value that enables easier comparison and prioritization of providers based on their respective claim likelihoods.

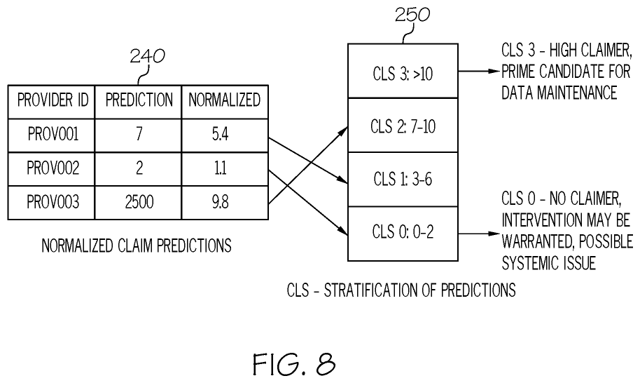

FIG. 8 is an illustrative example of claim likelihood score categories, according to some embodiments of the disclosure Referring to FIG. 8, claim likelihood score categories 250 (which may be referred to as a stratified claim likelihood score array) are considered, which organize the claim likelihood scores for each provider into categories based on their respective scores. The claim likelihood score categories 250 enable insurance providers and other stakeholders in the insurance industry to easily compare, prioritize, and analyze providers based on their claim likelihood scores, facilitating more effective resource allocation and risk management strategies.

The categories within the claim likelihood score categories 250, in some embodiments, adjust dynamically based on the population of providers or the distribution of claim likelihood scores. This dynamic adjustment ensures that the categories remain relevant and meaningful, even as the composition of the provider population or the range of claim likelihood scores changes over time. The dynamic adjustment takes the form of one or more of periodic recalibration, a sliding window, adaptive thresholding, trigger-based adjustments, clustering algorithms, or the like.

In some embodiments, the dynamic adjustment is performed utilizing periodic readjustment. Under periodic readjustment, the categories are recalibrated at regular intervals (e.g., quarterly, annually) to reflect the current distribution (or recent historical distribution) of claim likelihood scores. This involves redefining the category thresholds based on updated percentile ranks, mean, median, or standard deviation, to maintain a proper representation of the provider population and the scores.

In some embodiments, the dynamic adjustment is performed utilizing a sliding window technique. The sliding window technique is employed to ensure that the categories are defined based on the most recent data. This involves continuously updating the categories based on a fixed time window or a predefined number of data points, which allows the model to adapt to changes in the provider population and claim likelihood scores.

In some embodiments, the dynamic adjustment utilizes adaptive thresholding. Under adaptive thresholding, the categories are dynamically adjusted based on real-time data, where thresholds are updated as new data becomes available. This involves using adaptive algorithms or machine learning techniques to optimize category boundaries based on the observed distribution of claim likelihood scores and available resources, such as data maintenance resources.

In some embodiments, the dynamic adjustment utilize trigger-based adjustment. Under trigger-based adjustment, the categories are adjusted when certain predefined conditions or triggers are met, such as significant changes in the provider population, the distribution of claim likelihood scores, or a change in the availability of resources, such as data maintenance resources.

In some embodiments, the dynamic adjust utilizes machine-learning techniques. Under machine learning techniques, unsupervised or supervised machine learning techniques (such as clustering algorithms) are used to automatically identify and update the categories based on the patterns and trends in the claim likelihood scores. This helps ensure that the categories are both relevant and meaningful, even as the composition of the provider population or the range of claim likelihood scores changes over time.

In addition to the stratified categories, the claim likelihood score array 250 also identifies outliers, which are providers with abnormally high or low claim likelihood scores. These outliers are flagged within the array, drawing attention to providers that may warrant further investigation or analysis due to their atypical claim likelihood scores, which may help in identifying or guiding the allocation of data maintenance resources.

In some embodiments, an exemplary ARIMA (Autoregressive Integrated Moving Average) model is a statistical technique used for time-series analysis, forecasting, and prediction. The ARIMA model combines the autoregressive (AR), differencing (I), and moving average (MA) components to model a time-series data. The model can be represented by the following notation: ARIMA (p, d, q), where p represents the order of the autoregressive component, d represents the degree of differencing, and q represents the order of the moving average component.

The general equation for an ARIMA model can be expressed as:

y′(t)=c+Σ[φ(i)*y′(t−i)]+Σ[θ(j)*ε(t−j)]+ε(t)
where:
   y′(t) is the differenced time-series data at time t,
   c is a constant term,
   φ(i) represents the autoregressive coefficients,
   θ(j) represents the moving average coefficients,
   ε(t) is the error term at time t,
   i is the index for the autoregressive terms (1, 2, . . . , p),
   j is the index for the moving average terms (1, 2, . . . , q), and
   t represents the time index.

The autoregressive (AR) component of the ARIMA model captures the relationship between the current value of the time-series and its past values. The order of the AR component (p) indicates the number of past values (lags) considered in the model. The moving average (MA) component of the ARIMA model captures the relationship between the current value of the error term and its past values. The order of the MA component (q) indicates the number of past error terms (lags) considered in the model.

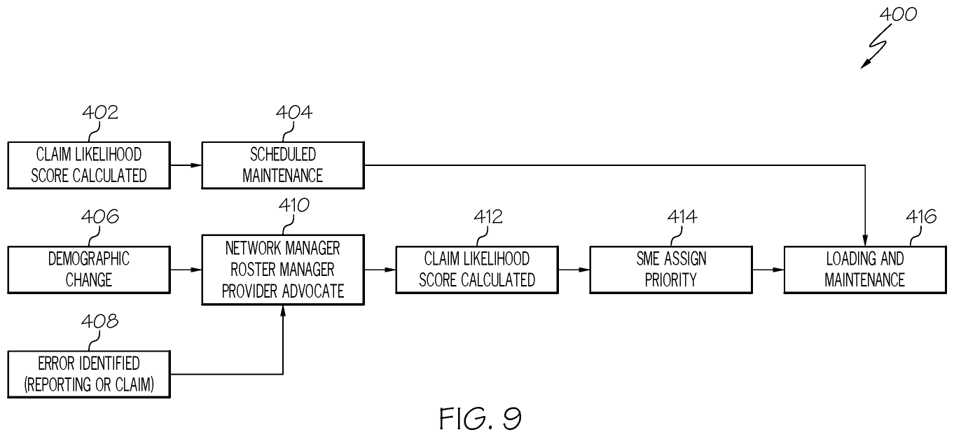

FIG. 9 is an illustrative example of a flowchart demonstrating a maintenance workflow, according to some embodiments of the disclosure. The maintenance workflow 400 enables for allocating resources effectively for data maintenance and loading operations based on claim likelihood scores and other relevant factors. The maintenance workflow 400 includes, in some embodiments, two routes or inputs to determine loading and maintenance operations.

At step 402, the method includes first calculating a claim likelihood score, utilizing the techniques and models described earlier. The claim likelihood score takes into account the historical claim information, time-series analysis models, and various other factors to generate a comprehensive assessment of the likelihood of future claims for a given provider.

At step 404, the method includes scheduling maintenance activities based on one or more prioritization criteria derived from the calculated claim likelihood scores. Prioritization may involve ranking providers or provider groups based on their respective claim likelihood scores or categories, with higher priority given to those with greater claim likelihoods or more significant deviations from the norm.

At step 416, the method includes performing loading and data maintenance activities based on the determined schedule and prioritization. These activities include updating provider data, addressing data inconsistencies, incorporating new claim information, and other tasks necessary to maintain the accuracy and relevance of the claim likelihood predictions.

At step 406, the method involves receiving a demographic change that may impact the claim likelihood score calculations. Demographic changes can include shifts in population characteristics, policyholder behavior patterns, or other factors that may influence the likelihood of claims within a given provider or provider group.

At step 408, the method includes identifying an error, such as reporting a specific claim, which may also influence the claim likelihood scores. Identifying and addressing errors is crucial for ensuring the accuracy and reliability of the claim likelihood predictions.

At step 410, these two pieces of information, demographic change and identified error, are utilized individually or together to inform one or more users and/or systems. In some embodiments, a system or team of users includes components such as a network manager, a roster manager, and a provider advocate, which collectively facilitate the integration and processing of new and updated information.

At step 412, the method includes calculating (or recalculating, as necessary) the claim likelihood score based on the updated demographic information and identified error. This step ensures that the recalculated claim likelihood scores accurately reflect the latest information and any necessary corrections.

At step 414, the method includes assigning a priority for data maintenance based on the updated claim likelihood score. The priority assignment involves reevaluating the ranking of providers or provider groups, adjusting maintenance schedules, and reallocating resources to address the most pressing maintenance needs.

At step 416, the method includes loading and data maintenance activities based on the assigned priority. These activities involve updating provider data, resolving data inconsistencies, incorporating new claim information, and other tasks needed to maintain the accuracy and relevance of the claim likelihood predictions.

In general, any process or operation discussed in this disclosure is understood to be computer-implementable, such as the process illustrated in FIGS. 2-9 are performed by one or more processors of a computer system as described herein. A process or process step performed by one or more processors is also referred to as an operation. The one or more processors are configured to perform such processes by having access to instructions (e.g., software or computer-readable code) that, when executed by one or more processors, cause one or more processors to perform the processes. The instructions are stored in a memory of the computer system. A processor is a central processing unit (CPU), a graphics processing unit (GPU), or any suitable type of processing unit.

A computer system, such as a system or device implementing a process or operation in the examples above, includes one or more computing devices. One or more processors of a computer system are included in a single computing device or distributed among a plurality of computing devices. One or more processors of a computer system are connected to a data storage device. A memory of the computer system includes the respective memory of each computing device of the plurality of computing devices.

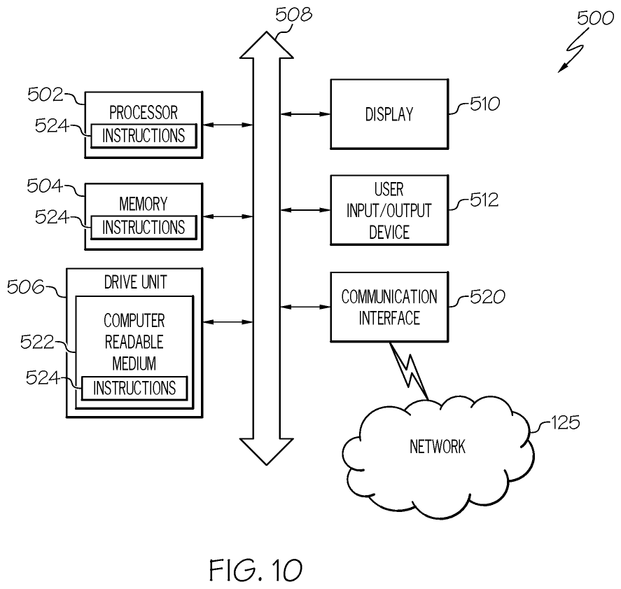

FIG. 10 illustrates an implementation of a computer system that executes techniques presented herein, according to some embodiments of the disclosure. The computer system 500 includes a set of instructions that are executed to cause the computer system 500 to perform any one or more of the methods or computer based functions disclosed herein. The computer system 500 operates as a standalone device or is connected, e.g., using a network, to other computer systems or peripheral devices.

Unless specifically stated otherwise, as apparent from the following discussions, it is appreciated that throughout the specification, discussions utilizing terms such as “processing,” “computing,” “calculating,” “determining”, analyzing” or the like, refer to the action and/or processes of a computer or computing system, or similar electronic computing device, that manipulate and/or transform data represented as physical, such as electronic, quantities into other data similarly represented as physical quantities.

In a similar manner, the term “processor” refers to any device or portion of a device that processes electronic data, e.g., from registers and/or memory to transform that electronic data into other electronic data that, e.g., is stored in registers and/or memory. A “computer,” a “computing machine,” a “computing platform,” a “computing device,” or a “server” includes one or more processors.

In a networked deployment, the computer system 500 operates in the capacity of a server or as a client user computer in a server-client user network environment, or as a peer computer system in a peer-to-peer (or distributed) network environment. The computer system 500 is also implemented as or incorporated into various devices, such as a personal computer (PC), a tablet PC, a set-top box (STB), a personal digital assistant (PDA), a mobile device, a palmtop computer, a laptop computer, a desktop computer, a communications device, a wireless telephone, a land-line telephone, a control system, a camera, a scanner, a facsimile machine, a printer, a pager, a personal trusted device, a web appliance, a network router, switch or bridge, or any other machine capable of executing a set of instructions (sequential or otherwise) that specify actions to be taken by that machine. In a particular implementation, the computer system 500 is implemented using electronic devices that provide voice, video, or data communication. Further, while the computer system 500 is illustrated as a single system, the term “system” shall also be taken to include any collection of systems or sub-systems that individually or jointly execute a set, or multiple sets, of instructions to perform one or more computer functions.

As illustrated in FIG. 5, the computer system 500 includes a processor 502, e.g., a central processing unit (CPU), a graphics processing unit (GPU), or both. The processor 502 is a component in a variety of systems. For example, the processor 502 is part of a standard personal computer or a workstation. The processor 502 is one or more processors, digital signal processors, application specific integrated circuits, field programmable gate arrays, servers, networks, digital circuits, analog circuits, combinations thereof, or other now known or later developed devices for analyzing and processing data. The processor 502 implements a software program, such as code generated manually (i.e., programmed).

The computer system 500 includes a memory 504 that communicates via bus 508. The memory 504 is a main memory, a static memory, or a dynamic memory. The memory 504 includes, but is not limited to computer-readable storage media such as various types of volatile and non-volatile storage media, including but not limited to random access memory, read-only memory, programmable read-only memory, electrically programmable read-only memory, electrically erasable read-only memory, flash memory, magnetic tape or disk, optical media and the like. In one implementation, the memory 504 includes a cache or random-access memory for the processor 502. In alternative implementations, the memory 504 is separate from the processor 502, such as a cache memory of a processor, the system memory, or other memory. The memory 504 is an external storage device or database for storing data. Examples include a hard drive, compact disc (“CD”), digital video disc (“DVD”), memory card, memory stick, floppy disc, universal serial bus (“USB”) memory device, or any other device operative to store data. The memory 504 is operable to store instructions executable by the processor 502. The functions, acts, or tasks illustrated in the figures or described herein are performed by the processor 502 executing the instructions stored in the memory 504. The functions, acts, or tasks are independent of the particular type of instruction set, storage media, processor, or processing strategy and are performed by software, hardware, integrated circuits, firmware, micro-code, and the like, operating alone or in combination. Likewise, processing strategies include multiprocessing, multitasking, parallel processing, and the like.

As shown, the computer system 500 further includes a display 510, such as a liquid crystal display (LCD), an organic light emitting diode (OLED), a flat panel display, a solid-state display, a cathode ray tube (CRT), a projector, a printer or other now known or later developed display device for outputting determined information. The display 510 acts as an interface for the user to see the functioning of the processor 502, or specifically as an interface with the software stored in the memory 504 or in the drive unit 506.

Additionally or alternatively, the computer system 500 includes an input/output device 512 configured to allow a user to interact with any of the components of the computer system 500. The input/output device 512 is a number pad, a keyboard, a cursor control device, such as a mouse, a joystick, touch screen display, remote control, or any other device operative to interact with the computer system 500.

The computer system 500 also includes the drive unit 506 implemented as a disk or optical drive. The drive unit 506 includes a computer-readable medium 522 in which one or more sets of instructions 524, e.g. software, is embedded. Further, the sets of instructions 524 embodies one or more of the methods or logic as described herein. The sets of instructions 524 resides completely or partially within the memory 504 and/or within the processor 502 during execution by the computer system 500. The memory 504 and the processor 502 also include computer-readable media as discussed above.

In some systems, computer-readable medium 522 includes the set of instructions 524 or receives and executes the set of instructions 524 responsive to a propagated signal so that a device connected to network 530 communicates voice, video, audio, images, or any other data over the network 530. Further, the sets of instructions 524 are transmitted or received over the network 530 via the communication port or interface 520, and/or using the bus 508. The communication port or interface 520 is a part of the processor 502 or is a separate component. The communication port or interface 520 is created in software or is a physical connection in hardware. The communication port or interface 520 is configured to connect with the network 530, external media, the display 510, or any other components in the computer system 500, or combinations thereof. The connection with the network 530 is a physical connection, such as a wired Ethernet connection, or is established wirelessly as discussed below. Likewise, the additional connections with other components of the computer system 500 are physical connections or are established wirelessly. The network 530 alternatively be directly connected to the bus 508.

While the computer-readable medium 522 is shown to be a single medium, the term “computer-readable medium” includes a single medium or multiple media, such as a centralized or distributed database, and/or associated caches and servers that store one or more sets of instructions. The term “computer-readable medium” also includes any medium that is capable of storing, encoding, or carrying a set of instructions for execution by a processor or that causes a computer system to perform any one or more of the methods or operations disclosed herein. The computer-readable medium 522 is non-transitory, and may be tangible.

The computer-readable medium 522 includes a solid-state memory such as a memory card or other package that houses one or more non-volatile read-only memories. The computer-readable medium 522 is a random-access memory or other volatile re-writable memory. Additionally or alternatively, the computer-readable medium 522 includes a magneto-optical or optical medium, such as a disk or tapes or other storage device to capture carrier wave signals such as a signal communicated over a transmission medium. A digital file attachment to an e-mail or other self-contained information archive or set of archives is considered a distribution medium that is a tangible storage medium. Accordingly, the disclosure is considered to include any one or more of a computer-readable medium or a distribution medium and other equivalents and successor media, in which data or instructions are stored.

In an alternative implementation, dedicated hardware implementations, such as application specific integrated circuits, programmable logic arrays, and other hardware devices, is constructed to implement one or more of the methods described herein. Applications that include the apparatus and systems of various implementations broadly include a variety of electronic and computer systems. One or more implementations described herein implement functions using two or more specific interconnected hardware modules or devices with related control and data signals that are communicated between and through the modules, or as portions of an application-specific integrated circuit. Accordingly, the present system encompasses software, firmware, and hardware implementations.

Computer system 500 is connected to the network 530. The network 530 defines one or more networks including wired or wireless networks. The wireless network is a cellular telephone network, an 802.10, 802.16, 802.20, or WiMAX network. Further, such networks include a public network, such as the Internet, a private network, such as an intranet, or combinations thereof, and utilizes a variety of networking protocols now available or later developed including, but not limited to TCP/IP based networking protocols. The network 530 includes wide area networks (WAN), such as the Internet, local area networks (LAN), campus area networks, metropolitan area networks, a direct connection such as through a Universal Serial Bus (USB) port, or any other networks that allows for data communication. The network 530 is configured to couple one computing device to another computing device to enable communication of data between the devices. The network 530 is generally enabled to employ any form of machine-readable media for communicating information from one device to another. The network 530 includes communication methods by which information travels between computing devices. The network 530 is divided into sub-networks. The sub-networks allow access to all of the other components connected thereto or the sub-networks restrict access between the components. The network 530 is regarded as a public or private network connection and includes, for example, a virtual private network or an encryption or other security mechanism employed over the public Internet, or the like.

In accordance with various implementations of the present disclosure, the methods described herein are implemented by software programs executable by a computer system. Further, in an example, non-limited implementation, implementations can include distributed processing, component/object distributed processing, and parallel processing. Alternatively, virtual computer system processing can be constructed to implement one or more of the methods or functionality as described herein.

Although the present specification describes components and functions that are implemented in particular implementations with reference to particular standards and protocols, the disclosure is not limited to such standards and protocols. For example, standards for Internet and other packet switched network transmission (e.g., TCP/IP, UDP/IP, HTML, and HTTP) represent examples of the state of the art. Such standards are periodically superseded by faster or more efficient equivalents having essentially the same functions. Accordingly, replacement standards and protocols having the same or similar functions as those disclosed herein are considered equivalents thereof.

It will be understood that the steps of methods discussed are performed in one embodiment by an appropriate processor (or processors) of a processing (i.e., computer) system executing instructions (computer-readable code) stored in storage. It will also be understood that the disclosure is not limited to any particular implementation or programming technique and that the disclosure is implemented using any appropriate techniques for implementing the functionality described herein. The disclosure is not limited to any particular programming language or operating system.

It should be appreciated that in the above description of example embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment. Thus, the claims following the Detailed Description are hereby expressly incorporated into this Detailed Description, with each claim standing on its own as a separate embodiment of this invention.

Furthermore, while some embodiments described herein include some but not other features included in other embodiments, combinations of features of different embodiments are meant to be within the scope of the invention, and form different embodiments, as would be understood by those skilled in the art. For example, in the following claims, any of the claimed embodiments can be used in any combination.

Furthermore, some of the embodiments are described herein as a method or combination of elements of a method that can be implemented by a processor of a computer system or by other means of carrying out the function. Thus, a processor with the necessary instructions for carrying out such a method or element of a method forms a means for carrying out the method or element of a method. Furthermore, an element described herein of an apparatus embodiment is an example of a means for carrying out the function performed by the element for the purpose of carrying out the invention.

In the description provided herein, numerous specific details are set forth. However, it is understood that embodiments of the invention are practiced without these specific details. In other instances, well-known methods, structures and techniques have not been shown in detail in order not to obscure an understanding of this description.

Thus, while there has been described what are believed to be the preferred embodiments of the invention, those skilled in the art will recognize that other and further modifications are made thereto without departing from the spirit of the invention, and it is intended to claim all such changes and modifications as falling within the scope of the invention. For example, any formulas given above are merely representative of procedures that may be used. Functionality may be added or deleted from the block diagrams and operations may be interchanged among functional blocks. Steps may be added or deleted to methods described within the scope of the present invention.

The above disclosed subject matter is to be considered illustrative, and not restrictive, and the appended claims are intended to cover all such modifications, enhancements, and other implementations, which fall within the true spirit and scope of the present disclosure. Thus, to the maximum extent allowed by law, the scope of the present disclosure is to be determined by the broadest permissible interpretation of the following claims and their equivalents, and shall not be restricted or limited by the foregoing detailed description. While various implementations of the disclosure have been described, it will be apparent to those of ordinary skill in the art that many more implementations and implementations are possible within the scope of the disclosure. Accordingly, the disclosure is not to be restricted except in light of the attached claims and their equivalents.

The present disclosure furthermore relates to the following aspects.

Example 1. A computer-implemented method for provider prioritization, comprising: receiving, by one or more processors, historical claim information from each of one or more providers; applying, by the one or more processors, a respective model to the historical claim information received from each of the one or more providers; determining, by the one or more processors, a respective expected number of claims for each of the one or more providers; normalizing, by the one or more processors, the respective expected number of claims for each of the one or more providers; determining, by the one or more processors, a respective claim likelihood score for each of the one or more providers; and ranking, by the one or more processors, one or more providers based on each provider's respective expected number of claims.

Example 2. The computer-implemented method of example 1, wherein the historical claim information is received from a plurality of providers, each provider of the plurality of providers belonging to a grouping of providers.

Example 3. The computer-implemented method of example 2, wherein each grouping of providers is associated with a respective model.

Example 4. The computer-implemented method of example 3, wherein each respective model is a time-series model.

Example 5. The computer-implemented method of example 4, wherein the time-series model is an Autoregressive Integrated Moving Average (ARIMA) model.

Example 6. The computer-implemented method of example 2, wherein each grouping of providers, collectively, defines a population, and wherein the determining of a claim likelihood score for each provider adjusts dynamically based at least in part on an attribute of the population.

Example 7. The computer-implemented method of example 6, wherein the attribute of the population is a number of providers contained within the population.

Example 8. The computer-implemented method of example 1, further comprising: categorizing, by the one or more processors, each of the one or more providers within a claim likelihood score category.

Example 9. The computer-implemented method of example 8, wherein one or more bounds of each category are pre-determined based on the historical claim information.

Example 10. The computer-implemented method of example 8, wherein one or more bounds of each category adjust dynamically based at least in part on a population of providers.

Example 11. A system for provider prioritization, comprising: a memory storing instructions; and a processor executing the instructions to perform a process including: receiving historical claim information from each of one or more providers; applying a respective model to the historical claim information received from each of the one or more providers; determining a respective expected number of claims for each of the one or more providers; normalizing the respective expected number of claims for each of the one or more providers; determining a respective claim likelihood score for each of the one or more providers; and ranking one or more providers based on each provider's respective expected number of claims.

Example 12. The system of example 11, wherein historical claim information is received from a plurality of providers, each provider of the plurality of providers belonging to a grouping of providers.

Example 13. The system of example 12, wherein each grouping of providers is associated with a respective model.

Example 14. The system of example 13, wherein each respective model is a time-series model.

Example 15. The system of example 14, wherein the time-series model is an Autoregressive Integrated Moving Average (ARIMA) model.

Example 16. The system of example 12, wherein each grouping of providers, collectively, defines a population, and wherein the determining of a claim likelihood score for each provider adjusts dynamically based at least in part on an attribute of the population.

Example 17. The system of example 16, wherein the attribute of the population is a number of providers contained within the population.

Example 18. The system of example 11, further comprising: categorizing each of the one or more providers within a claim likelihood score category.

Example 19. The system of example 18, wherein one or more bounds of each category adjust dynamically based at least in part on a population of providers.

Example 20. A computer implemented method for provider prioritization, comprising: receiving, by one or more processors, historical claim information from a plurality of providers, each provider of the plurality of providers belonging to a grouping of providers; applying, by the one or more processors, an Autoregressive Integrated Moving Average (ARIMA) model to the historical claim information from each of the plurality of providers, wherein each grouping of providers is associated with a respective model; determining, by the one or more processors, a respective expected number of claims for each of the plurality of providers; normalizing, by the one or more processors, the respective expected number of claims for each of the plurality of providers; determining, by the one or more processors, a respective claim likelihood score for each of the plurality of providers; categorizing, by the one or more processors, each of the plurality of providers within a claim likelihood score category; and prioritizing, by the one or more processors, one or more of the plurality of providers based on each provider's respective expected number of claims, wherein one or more bounds of each category adjust dynamically based at least in part on a population of providers.

What is claimed is:

1. A computer-implemented method for provider prioritization, comprising:
receiving, by one or more processors, historical claim information from each of one or more providers;
applying, by the one or more processors, a respective model to the historical claim information received from each of the one or more providers;
determining, by the one or more processors, a respective expected number of claims for each of the one or more providers;
normalizing, by the one or more processors, the respective expected number of claims for each of the one or more providers;
determining, by the one or more processors, a respective claim likelihood score for each of the one or more providers; and
ranking, by the one or more processors, one or more providers based on each provider's respective expected number of claims.

2. The computer-implemented method of claim 1, wherein the historical claim information is received from a plurality of providers, each provider of the plurality of providers belonging to a grouping of providers.

3. The computer-implemented method of claim 2, wherein each grouping of providers is associated with a respective model.

4. The computer-implemented method of claim 3, wherein each respective model is a time-series model.

5. The computer-implemented method of claim 4, wherein the time-series model is an Autoregressive Integrated Moving Average (ARIMA) model.

6. The computer-implemented method of claim 2, wherein each grouping of providers, collectively, defines a population, and wherein the determining of a claim likelihood score for each provider adjusts dynamically based at least in part on an attribute of the population.

7. The computer-implemented method of claim 6, wherein the attribute of the population is a number of providers contained within the population.

8. The computer-implemented method of claim 1, further comprising: categorizing, by the one or more processors, each of the one or more providers within a claim likelihood score category.

9. The computer-implemented method of claim 8, wherein one or more bounds of each category are pre-determined based on the historical claim information.

10. The computer-implemented method of claim 8, wherein one or more bounds of each category adjust dynamically based at least in part on a population of providers.

11. A system for provider prioritization, comprising:
a memory storing instructions; and
a processor executing the instructions to perform a process including:
receiving historical claim information from each of one or more providers;
applying a respective model to the historical claim information received from each of the one or more providers;
determining a respective expected number of claims for each of the one or more providers;
normalizing the respective expected number of claims for each of the one or more providers;
determining a respective claim likelihood score for each of the one or more providers; and
ranking one or more providers based on each provider's respective expected number of claims.

12. The system of claim 11, wherein historical claim information is received from a plurality of providers, each provider of the plurality of providers belonging to a grouping of providers.

13. The system of claim 12, wherein each grouping of providers is associated with a respective model.

14. The system of claim 13, wherein each respective model is a time-series model.

15. The system of claim 14, wherein the time-series model is an Autoregressive Integrated Moving Average (ARIMA) model.

16. The system of claim 12, wherein each grouping of providers, collectively, defines a population, and wherein the determining of a claim likelihood score for each provider adjusts dynamically based at least in part on an attribute of the population.

17. The system of claim 16, wherein the attribute of the population is a number of providers contained within the population.

18. The system of claim 11, further comprising: categorizing each of the one or more providers within a claim likelihood score category.

19. The system of claim 18, wherein one or more bounds of each category adjust dynamically based at least in part on a population of providers.

20. A computer implemented method for provider prioritization, comprising:
receiving, by one or more processors, historical claim information from a plurality of providers, each provider of the plurality of providers belonging to a grouping of providers;
applying, by the one or more processors, an Autoregressive Integrated Moving Average (ARIMA) model to the historical claim information from each of the plurality of providers, wherein each grouping of providers is associated with a respective model;
determining, by the one or more processors, a respective expected number of claims for each of the plurality of providers;
normalizing, by the one or more processors, the respective expected number of claims for each of the plurality of providers;
determining, by the one or more processors, a respective claim likelihood score for each of the plurality of providers;
categorizing, by the one or more processors, each of the plurality of providers within a claim likelihood score category; and
prioritizing, by the one or more processors, one or more of the plurality of providers based on each provider's respective expected number of claims,
wherein one or more bounds of each category adjust dynamically based at least in part on a population of providers.
